(19) United States
(12) Patent Application Publication
(10) Pub. No.: US 2004/0161426 A1
(43) Pub. Date: Aug. 19, 2004

(54) Use of IL-6 antagonists in combination with steroids to enhance apoptosis

(76) Inventors: Mohit Trikha, Malvern, PA (US); Mohamed Zaki, Audubon, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/759,674

(22) Filed: Jan. 16, 2004

Related U.S. Application Data
(60) Provisional application No. 60/444,843, filed on Feb. 4, 2003.

(51) Int. Cl. A61K 39/395; A61K 31/573

(52) U.S. Cl. 424/145.1; 514/171

(57) ABSTRACT

Methods for the use of antibodies directed toward IL-6, including specified portions or variants, specific for at least one Interleukin-6 (IL-6 also known as interferon β2)) protein or fragment thereof, in combination with steroids for the treatment of proliferative diseases such as cancer which are amenable to treatment by apoptosis inducing agents.

### BACKGROUND OF THE INVENTION

1. Field of the Invention

The present invention relates to a method of using antibodies to treat pathological processes associated with proliferative diseases, such as cancer, by promoting the process of apoptosis. The invention more specifically relates to methods for the use of antibodies directed toward IL-6, including specified portions or variants, specific for at least one Interleukin-6 (IL-6 also known as interferon β2)) protein or fragment thereof in combination with steroids for the treatment of proliferative diseases such as cancer which are amenable to treatment by apoptosis inducing agents.

2. Background

The need to develop more effective and less toxic therapeutic regiments to treat malignant diseases is becoming a major focus of cancer research. Specific factors such as cytokines that are either produced by the tumor cells or present in the tumor environment can contribute to both tumor growth and resistance to standard therapy. Targeted therapy using monoclonal antibodies towards those factors or towards specific receptors expressed by tumor cells might be the most effective way to treat cancer. Monoclonal antibodies have become the most rapidly expanding class of pharmaceuticals for treating a wide variety of human diseases, including cancer. Although antibodies have yet to achieve the ultimate goal of curing cancer, many innovative approaches stand poised to improve the efficacy of antibody-based therapies. (Carter Nature Rev Cancer (1) 118-29, 2001.

Cytokine IL-6

IL-6 (interleukin 6) is a 22-27 kDa secreted glycoprotein formerly known as monocyte-derived human B-cell growth factor, B-cell stimulatory factor 2, BSF-2, interferon beta-2, and hybridoma growth factor, which has growth stimulatory and proinflammatory activities (Hirano et al. Nature 324: 73-76, 1986).

IL-6 belongs to the granulocyte colony-stimulating factor (G-CSF) and myelomonocytic growth factor (MGF) family which includes leukemia inhibitory factor (LIF), oncostatin M (OSM), ciliary neurotropic factor (CNTF), cardiotropin-1 (CT-1), IL-1, and IL-11. IL-6 is produced by an array of cell types, most notably antigen presenting cells, T cells and B cells. IL-6-type cytokines all act via receptor complexes containing a common signal transducing protein, gp130 (formerly IL-6Rbeta). However, whereas IL-6, IL-11, CT-1, and CNTF bind first to specific receptor proteins which subsequently associate with gp130, LIF and OSM bind directly to a complex of LIF-R and gp130. The specific IL-6 receptor (IL-6R or IL-6alpha, gp80, or CD126) exists in either membrane bound or soluble forms (slL-6R, a 55 kD form), which are both capable of activating gp130.

Several agents are known to induce the expression of IL-6 including IL-1, IL-2, TNFα, IL-4, IFNα, oncostatin and LPS. IL-6 is involved in diverse activities such as B and T cell activation, hematopoiesis, osteoclast activity, keratinocyte growth, acute phase protein synthesis, neuronal growth and hepatocyte activation (Hirano et al. Int. Rev. Immunol;16(3-4):249-84, 1998).

Although IL-6 is involved in many pathways, IL-6 knockout mice have a normal phenotype, they are viable and fertile, and show slightly decreased number of T cells and decreased acute phase protein response to tissue injury (Kopf M et al. Nature: 368:339-42, 1994). In contrast, transgenic mice that over-express cerebral IL-6 develop neurologic disease such as neurodegeneration, astrocytosis, cerebral angiogenesis, and these mice do not develop a blood brain barrier (Campbell et al. PNAS 90: 10061-10065, 1993).

The Role of IL-6 in Cancer

IL-6 is implicated in the pathophysiology of several malignant diseases by a variety of mechanisms. IL-6 is hypothesized to be a causative factor in cancer-related morbidity such as asthenia, cachexia and bone resorption. Tumor-induced cachexia (Cahlin et al. (2000) Cancer Res; 60(19):5488-9), bone resorption and associated hypercalcemia were found to be diminished in IL-6 knockout mice (Sandhu et al. 1999). Cancer-associated depression, and cerebral edema secondary to brain tumors have also been associated with high levels of IL-6 (Musselman et al. Am J Psychiatry.;158(8):1252-7, 2001).

Experimental results from a number of in vitro and in vivo models of various human cancers have demonstrated that IL-6 is a therapeutic target for inhibition. IL-6 can induce proliferation, differentiation and survival of tumor cells, promote apoptosis (Jee et al. Oncogene 20: 198-208,2001), and induce resistance to chemotherapy (Conze et al. Cancer Res 61: 8851-8858, 2001).

Multiple myeloma is malignancy involving plasma cells. IL-6 is known to enhance proliferation, differentiation and survival of malignant plasma cells in multiple myeloma (MM) through an autocrine or a paracrine mechanism that involves the inhibition of apoptosis of the malignant cells. Accordingly, blocking of IL-6 has been postulated to be an effective therapy (Anderson et al. Hematology:147-165, 2000). Both in vitro experiments (Tassone, P. et al. Int. J. Oncol. 21(4): 867-873, 2002) and clinical trials have been performed (Bataille et al. (1995) Blood; 86(2):685-91 and Van Zaanen, et al. (1996) J Clin Invest 98: 1441-1448) and the results indicate that IL6 blockade has demonstrable effect on cancer cell growth.

Specific factors such as cytokines that are either produced by the tumor cells or present in the tumor environment can contribute to both tumor growth and resistance to standard therapy. Cytokines, such as IL-6, that bind to cell surface receptors and either modulate the immune response or inhibit some of the death signaling domains, render the cells resistant to steroids or chemotherapy induced cell death (Fehniger et al., Cytokine Growth Factor Rev 13:169-83, 2002).

Steroids Induce Apoptosis

Apoptosis is a form of programmed cell death that occurs under numerous developmental and physiological conditions that require the selective elimination of cells from tissues and organs without the production of an inflammatory response. The initiation of apoptosis is controlled bythe balance between death and life signals perceived by the cell. The apoptotic response by cells perceiving a death stimulus includes: a reduction in cell volume, compaction of intracellular organelles, chromatin condensation, and the generation of apoptotic bodies which contain degraded cellular components. This mode of death is in contrast to lytic mechanisms which releases cell contents into the surrounding environment. Apoptotic bodies are often engulfed by neighboring cells or macrophages, preventing the occurrence of an inflammatory response in the region of the dying cells.

Dexamethasone, a steroid drug, is a catabolic effector molecule that initiates the apoptotic process and causes what is termed glucocorticoid-induced apoptosis in rodent and human lymphocytes. These cells respond to dexamethasone with cell growth arrest, chromatin condensation, cell shrinkage, and the selective degradation of DNA, RNA, and protein. The response is dependent on the presence of functional glucocorticoid receptors and requires gene expression. The fragmentation of DNA and its associated cell shrinkage is an irreversible commitment to cell death (Cidlowski et al., Recent Prog Horm Res (51) 457-90, 1996).

Monoclonal Antibodies to IL-6

Murine monocolonal antibodies to IL-6 are known as in, for example, U.S. Pat. No. 5,618,700. U.S. Pat. No. 5,856,135 discloses reshaped human antibodies to human IL-6 derived from a mouse monoclonal antibody SK2 in which the complementary determining regions (CDR's) from the variable region of the mouse antibody SK2 are transplanted into the variable region of a human antibody and joined to the constant region of a human antibody.

Another murine IL-6 monoclonal antibody referred to as CLB-6/8 capable of inhibiting receptor signaling was reported (Brakenhoff et al, J. Immunol. (1990) (145:561). A chimerized form of this antibody called cCLB8 was constructed (Centocor, Malvern, Pa.) and has been given to multiple myeloma patients (Van Zaanen, et al. 1996 supra). The chimerized antibody and the method of making the resulting antibody from the murine antigen binding domains has been fully described in the applicants' copending application U.S. Ser. No. 60/332,743 hereby incorporated by reference into the present application.

Analysis of patient serum samples prior to and after cCLB8 administration showed that circulating levels of both sIL6R and sgp130 were high in these patients and remained unchanged by the treatment despite total blockage of serum IL-6 activity (VanZaanen, et al. Leukemia Lymphoma 31(506): 551-558, 1998.)

B-E8 is a murine mAb to IL-6 manufactured by Diaclone, France which has also undergone clinical evaluation. B-E8 mAb demonstrated effectiveness in treating B-lymphoproliferative disorders (Haddad et al 2001). In AIDS associated lymphoma, this anti-IL-6 mAb had a clear effect on lowering lymphoma-associated fever and loss of weight due to cachexia, thereby improving indices of the quality of life for those patients (Emilie et al. (1994) Blood 84(8):2472-9). B-E8 has also been us renal carcinoma patients. Metastatic renal cell carcinoma (RCC) is frequently associated with high levels of IL-6 and it is accompanied by paraneoplastic symptoms. B-E8 treatment had a significant reduction in the paraneoplastic syndrome in three RCC patients (Blay et al., Int J Cancer; 72(3): 424-30, 1997). In another published clinical trial, six patients with RCC were treated with B-E8 (Legouffe et al. (1994) Clin Exp Immunol. 98(2): 323-9). All of the treated patients demonstrated a loss of symptoms generally attributable to IL-6 overproduction following B-E8 treatment.

The clinical experience with anti-IL6 Mabs has been limited to date. However, several in vitro and murine models of various human tumors have been used to demonstrate that anti-IL-6 Mabs have the potential to impact tumor cell survival and disease progression including: inhibiting growth of human brain tumor cells (Goswami et al. (1998) J Neurochem 71: 1837-1845) or tumors (Mauray et al. 2000), human renal carcinoma tumors and serum calcium concentrations (Weisglass et al. (1995) Endocrinology 138(5):1879-8), and human hormone refractory prostate tumor xenografts (Smith et al. (2001) Prostate; 48(1):47-53). In one reported case, (B. Klein et al, Blood, 78: 1198-1204 (1991), a patient with plasma cell leukemia who had been treated unsuccessfully with cytotoxic chemotherapy (VAD regimen), was treated with anti-IL-6 therapy followed by treatment with dexamethasone to limit the effects of a putative immunization. The anti-IL-6 Mabs blocked myeloma cell proliferation in vivo for 45 days.

In summary, IL-6 is a pleiotropic cytokine that can promote the pathogenesis of malignant diseases through several mechanisms. Preclinical data have shown that IL-6 is a survival, proliferation and differentiation factor in several types of tumors including renal cancer and prostate cancer. IL-6 also plays a major role in development of cancer related morbidity such as cachexia, bone resorption and depression and it can cause resistance to chemotherapy by inducing MDR1 gene expression. Clinical data have shown that elevated levels of IL-6 contribute to the malignant process in several diseases and preliminary clinical trials have shown some disease attenuating activity of anti-IL-6 Mabs.

There is a need for agents capable of limiting the growth, survival, and metastatic potential of tumor cells, particularly renal carcinoma and hormone refractory prostate carcinoma. Apoptosis describes a particular sequence of events which eliminates viable cells from a tissue. The induction of apoptosis, therefore, in tumor tissue is desirable in so far as it reduces the tumor mass while preventing the release of tumor derived toxins which contribute to cancer related side effects. While steroid drugs promote apoptosis, IL6 protects against apoptosis specifically of cancer cells.

Therefore, it would be extremely desirable to have cancer treatment regimens that both induce apoptosis of unwanted pathogenic cells, such as malignant cells, and provide protection against the undesirable effects of excess IL-6 on tumor growth and resistance to apoptotic and other chemotherapy agents while at the same time ameliorating the ancillary and detrimental effects of excess endogenously produced IL-6 on the host such as asthenia, cachexia, and bone resorption.

### SUMMARY OF THE INVENTION

This invention is a method of treating proliferative diseases amenable to treatment by apoptosis inducing agents in a patient in need of such treatment, which comprises co-administering an agent capable of inducing apoptosis and an IL-6 antagonist. In a preferred embodiment the apoptotic agent is a corticosteroid, most preferably dexamethasone, and the IL-6 antagonist is a monoclonal antibody specific for IL-6.

In one aspect, the IL-6 antagonist is an anti-IL-6 antibody. In this respect, the invention relates to a method of using antibodies directed toward IL-6, including specified portions or variants, specific for at least one Interleukin-6 (IL-6 also known as Interferon β2)) protein or fragment thereof, to augment the therapeutic effect of corticosteroid therapy. Such anti-IL-6 antibodies can act through their ability to prevent the interaction of IL-6 with membrane bound receptor in a manner that prevents events associated with the initiation or progression of cancer tissue including events leading to enhanced tumor cell survival, tumor growth, and metastatic spread. In a particular embodiment, the anti-IL-6 antibody used in combination with the steroid is one that specifically binds IL-6 in a manner that prevents its action systemically and locally. The antibodies may bind to IL6 creating a long-lived complex incapable of activating membrane bound receptor, such as gp130, in any tissue accessible by the complex through normal circulatory mechanisms. The method of the present invention thus employs antibodies having the desirable neutralizing property which makes them ideally suited for therapeutic and preventative treatment of metastatic disease states associated with various forms of cancer in human or nonhuman patients.

Accordingly, the present invention is directed to a method of treating a disease or condition which as a component involves the prolonged survival of unwanted cell types, such as malignant cells, in a patient in need of such treatment which comprises administering to the patient an amount of a neutralizing IL-6 antibody to enhance apoptosis.

### BRIEF DESCRIPTION OF THE DRAWINGS

FIG. 1A-C. Scatter diagrams showing the data points for Tdt+ RPMI 8662 cells (terminal deoxynucleotidylexotransferase)-mediated dUTP-FITC nick end labeled) which represent cells actively undergoing apoptosis when treated with dexamethasone.

FIG. 1A shows the level of apoptosis (45%) in a representative experiment for cells treated with dexamethasone. FIG. 1B shows the level of apoptosis (20%) when IL-6 is added to cells treated with the same concentration of dexamethasone as in 1A. FIG. 1C shows the level of apoptosis (60%) in cells treated with dexamethasone and IL6 as in 1B but where anti-IL6 antibody is also present.

### DETAILED DESCRIPTION OF THE INVENTION

Two types of steroid hormones are synthesized in the adrenal cortex: corticosteroids and androgens. Corticosteroids (glucocorticoids and mineralocorticoids) are catabolic while the androgens are generally anabolic. Glucocorticoids, as represented by hydrocortisone, are so-named because of their role in regulating carbohydrate-metabolism. Mineralcorticords, as represented by aldosterone, regulate electrolyte balance. In addition to these functions, corticosteroids afford the individual (human or animal) the ability to cope with stressful environmental conditions or noxious stimuli. The daily output of corticosteroids by the adrenals can rise as much as 10-fold in response to stress. Therefore, the pharmacological agents that are corticosteroid analogs have therapeutic effects that are the side effects on physiological processes of the natural regulators of metabolic processes. For example, the anti-inflammatory and immunosuppressive actions of corticosteroids are one of the major therapeutic uses of drugs that mimic glucocorticoids, such as prednisone or dexamethasone. As understood herein, the term “steroid” refers to glucocorticoids or therapeutic agents which are analogs of or mimetics of glucocorticoids.

The understanding of the all the interactions that lead to lymphocytopenia in some situations and increased production of lymphoid tissue on the other hand in response to elevated or exogenous steroid is still incomplete. However, it is common practice to give steroids in the course of treating lymphoid malignancies. Likewise, suppression of inflammation is of enormous clinical benefit in a variety of instances as is the immunosuppressive effect of steroids. Steroids block or inhibit production and release of prostaglandins and leukotrienes, as well as the inflammatory cytokines; IL-1, IL-6, and TNFalpha, and acute phase reactants from macrophages and monocytes, endothelial cells, and fibroblasts. In addition, steroids reduce the elaboration of surface adhesion molecules on endothelial cells, the release of histamine by basophils, and the release of additional cytokines (IL-2, IL-3, and IFNgamma) from lymphocytes and suppress growth factor induced proliferation of fibroblasts.

Corticosteroids inhibit the inflammatory response to a variety of inciting agents and probably delay or slow healing. They transiently inhibit the edema, fibrin deposition, capillary dilation, leukocyte migration, capillary proliferation, fibroblast proliferation, deposition of collagen, and scar formation associated with inflammation. There is no generally accepted explanation for the mechanism of action of ocular corticosteroids. However, corticosteroids are thought to act by the induction of phospholipase A2 inhibitory proteins, collectively called lipocortins. It is postulated that these proteins control the biosynthesis of potent mediators of inflammation such as prostaglandins and leukotrienes by inhibiting the release of their common precursor arachidonic acid. Arachidonic acid is released from membrane phospholipids by phospholipase A2. Corticosteroids are capable of producing a rise in intraocular pressure.

In effect, the hypothalamic-pituitary-adrenal axis (HPA axis) communicates with the immune system and it has been suggested that the action of steroids is to protect against the life-threatening activity of the cytokine “storm” which can accompany severe infection, trauma, or cancer. As such, steroids and IL6 are on opposing sides in the balancing act.

Use of steroids is not nontoxic. The toxic effects of therapeutic use of steroids are of two categories: those resulting from the use of supraphysiological levels of the hormone and those resulting from withdrawal from the effects of these above normal levels. Both types of side effects are potentially lethal. Prolonged therapy can lead to fluid and electrolyte abnormalities, hypertension, hyperglycemia, increased susceptibility to infection, osteoporosis, myopathy, behavioral disturbances, cataracts, growth arrest, and the physiological changes including adipose redistribution and hirsutism.

The effects of steroids on bone and calcium distribution are due to decreased activity of osteoblasts, decreased Ca2+ absorption in the gut, and increased PTH production. These effects are actually compounded by the effects of IL6 which promotes osteoclast activity as well as PTH release resulting in hypercalcemia and therefore the risk of thrombotic events.

The most frequent problem with withdrawal from steroid therapy is recurrence of the underlying condition, which may include graft rejection is the case of a transplant. Other complications include acute renal insufficiency as a consequences of HPA axis suppression. Recovery from steroid withdrawal may take from weeks to a year or longer.

Besides treating adrenal insufficiency syndromes and post-menopausal estrogen loss, estrogen loss due to ovariectomy or total hysterectomy, steroid therapy may be administered to treat non-endocrine disorders which are immune-mediated or require control of inflammatory mediators such as rheumatic disorders, renal diseases, allergic disease, bronchial asthma, ocular diseases, skin diseases, gastrointestinal diseases, hepatic diseases, malignancies, cerebral edema (due to parasites or neoplasms), hemolytic anemias, and stroke and spinal cord injury.

Other conditions or diseases wherein steroid therapy is used are exemplified by, but not limited to adrenal hyperplasia, adrenocortical insufficiency, alopecia areata, acquired hemolytic anemia, hypoplastic anemia (congenital), ankylosing spondylitis , gouty and psoriatic arthritis, berylliosis, bronchial asthma, bursitis, allergic and vernal conjunctivitis, cerebral palsy, chorioretinitis, choroiditis, chronic obstructive lung disease, ulcerative colitis, collagen disease, allergic conjunctivitis and corneal marginal ulcers, atopic and contact dermatitis, herpetiformis bullous dermatitis, seborrhea, edema due to lupus erythematosus, lupus nephritis, cerebral edema, regional enteritis, epicondylitis, erythroblastopenia, granuloma annulare, herpes zoster ophthalmicus, inflammation of the eye including iridocyclitis, iritis, keloids, keratitis, laryngeal edema, lichen planus, lichen simplex chronicus, Loeffler's syndrome, lupus erythematosus discoides, lupus erythematosus, systemic, meningitis, tuberculous, myositis, mycosis fungoides, necrobiosis lipoidica diabeticorum, nephrotic/nephritic syndrome, anti-glomerular basement membrane nephritis, ophthalmia, optic neuritis, synovitis of osteoarthritis, pemphigus, psoriatic, idiopathic thrombocytopenic purpura, rheumatic carditis, rheumatoid arthritis, rheumatoid arthritis, chronic rhinitis, sarcoidosis, scleroderma, serum sickness, shock, Stevens-Johnson syndrome, tenosynovitis, takayasuds arteritis, Wegener's granulomatosis, acute nonspecific thrombocytopenia, thyroiditis, trichinosis with myocardial involvement, trichinosis with neurologic involvement, tuberculosis, urticaria, uveitis.

Steroid therapy may also be used in conjunction with an organ or tissue transplant, such as a bone marrow transplant or a multiple organ transplant. In certain aspects of the invention, the steroid is administered at a high dose and/or over a long period of time.

Cancers arising from immune cell abnormalities are commonly treated with steroid drugs. These include myeloid cancers such as multiple myeloma, and myelogenous leukemia (CML), as well as lymphocytic leukemia (CLL and ALL) and lymphomas, particularly Non-Hodgkin's Lymphoma (NHL). Other cancers forming solid tumors including prostate, and breast cancers can be treated with the method of the present invention and, due to its minimally toxic nature, in combination with other agents and where adjunctive forms of therapy are being practiced, such as radiation therapy.

Other “solid tumor” forming cancers, include, but are not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, pancreatic or gastric adenocarcinoma, human papilomavirus associated cervical intraepithelial neoplasia, and hepatoma.

A secondary tumor, a metastasis, is a tumor which originated in a primary site in the body and spread to a distant organ. The common routes for metastasis are direct growth into adjacent structures, spread through the vascular or lymphatic systems, and tracking along tissue planes and body cavities with, for example, peritoneal fluid or cerebrospinal fluid. Secondary hepatic tumors are one of the most common causes of death in cancer patients and are by far and away the most common form of liver tumor. Although virtually any malignancy can metastasize to the liver, tumors which are most likely to spread to the liver include: cancer of the stomach, colon, and pancreas; melanoma; tumors of the lung, oropharynx, and bladder; Hodgkin's and non-Hodgkin's lymphoma; tumors of the breast, ovary, and prostate. Secondary lung, brain, and bone tumors are common to advanced stage breast, prostate and lung cancers. Any cancer may metastasize to bone, but metastases from carcinomas are the most common, particularly those arising in the breast, lung, prostate, kidney, and thyroid. Carcinoma of the lung is very commonly accompanied by hematogenous metastatic spread to the liver, brain, adrenals, and bone and may occur early, resulting in symptoms at those sites before obvious pulmonary symptom. Metastases to the lungs are common from primary cancers of the breast, colon, prostate, kidney, thyroid, stomach, cervix, rectum, testis, and bone and from melanoma. Each one of the above-named secondary tumors may be treated by the antibodies of the present invention.

Bone Loss

Bone loss is associated with and/or caused by steroid therapy as are high levels of circulating IL6 in cancer patients. In addition to bone loss due to aging and estrogen deficiency, patients of all ages, both sexes, and all races are susceptible to steroid-induced bone loss. Administration of glucocorticoids and steroids is the third most common cause of osteoporosis. Steroid-induced bone loss usually affects the cortical and cancellous bone of the axial skeleton. Between 30% and 50% of individuals taking steroids for more than 6 months will develop osteoporosis. The rate of bone loss is very rapid in the initial year of therapy, with as much as 20% of the bone lost in the first year. Doses exceeding 7.5 mg/day of prednisone can cause significant loss of trabecular bone in most people.

Studies in mice administered glucocorticoids suggests that steroid- induced bone loss is due to decreased bone formation which results from higher numbers of apoptotic/dead osteoclasts and osteoblasts. Lesser numbers of these cells could account for changes seen with glucocorticoid-induced bone disease. A decrease in osteoblast and osteocyte cell number due to death/apoptosis has also been demonstrated in patients who have glucocorticoid-induced osteoporosis (Weinstein et al., 1998).

Despite the current understanding and the considerable amount of research in this area, bone loss and osteoporosis remain significant medical and economic problems. Therefore, methods of reducing or preventing bone loss, for example by reducing or preventing apoptosis of osteocytes and osteoblasts, would represent a significant advance in the art.

Thus a particularly advantageous aspect of the present invention is to allow the treatment of disease with steroid therapy while preventing or ameliorating the effects on bone, such as bone resorption and concomitant hypercalcemia.

Methods of Evaluating Apoptotic Activity

Many events occur during the process of apoptosis that can be assayed to determine if cells are undergoing apoptosis and/or the extent of apoptosis. Nuclear matrix proteins (NMP) have been shown to dissociate and solubilize during apoptosis, which likely accounts for certain morphological changes seen in the nucleus of an apoptotic cell. Thus, detection of release of one or more NMP, particularly in a degraded state, such as lamin, can be used to assess apoptosis. Morphological measurements related to loss of nuclear structure and chromosome condensation into discrete balls are other markers of apoptosis. Degradation of the DNA produces 180 to 200 bp fragments that can be visualized as a DNA ladder by agarose or acrylamide gel electrophoresis. These nucleosomal fragments can also be labeled radioactively, flourescently, or with enzymes that can catalyze a color producing reaction. The fragments that possess free 3′ hydroxyl groups can be labeled using terminal deoxynucleotidyl transferase, and those lacking the ternminal 3′ hydroxyl group can be labeled using the Klenow fragment of E. coli DNA polymerase I.

In addition to nuclear changes, plasma and mitochondrial membrane perturbations occur early in apoptosis. Phosphatidylserine, which is restricted to the inner surface of the plasma membrane bilayer in normal cells, is externalized to the outer plasma. Phosphatidylserine on the outer surface of the plasma membrane can be detected by annexin, which has a high affinity for phosphatidylserine (Martin et al., 1995), or by anti-phosphatidylserine antibodies. Furthermore, certain dyes that are excluded from viable cells, such as trypan blue and propidium iodide, stain apoptotic cells due to these membrane perturbations.

Release of the cytosolic enzymes such as lactate dehydrogenase or loss of mitochondrial function, such as by measuring electron transfer to a dye, MTT ([3-(4,5-dimethylthiazol-2-yl)2,5-diphenyltetrazolium bromide] can be measured spectrophotometrically.

Among the assays currently used to monitor apoptosis, the most common are visual methods, such as light or electron microscopy to determine cellular morphology, vital dye exclusion, nuclear staining with fluorescent dyes such as propidium iodide, acridine orange, bisbenzimide (Hoechst 33258 and 33342) and green fluorescent protein (GFP), indirect methods such as fluorescence-activated cell sorting (FACS) of fluorescently labeled cells, assays for the release of the cytosolic enzyme lactate dehydrogenase, the MTT/XTT assay, detection of binding of annexin V or anti-phosphatidylserine antibodies, detection of DNA fragmentation, detection of the release of soluble nuclear matrix proteins, such as nuclear matrix protein A, from cells, detection of the loss of lamins from the nuclear envelope and detection of free nucleosomes. Additionally, in certain instances these assays are combined, such as determining the binding of annexin V or anti-phosphatidylserine antibodies in conjunction of dye exclusion, such as propidium iodide. Annexin V labeled with either FITC or biotin, as well as a monoclonal anti-phosphatidylserine antibody, are available. Kits for the labeling and detection of these DNA fragments, four monoclonal antibodies against nuclear matrix proteins, as well as a kit for detecting soluble nuclear matrix proteins, anti-laminin antibodies are available, as are kits for detecting free nucleosomes. Many of these reagents are available commercially from Oncogene Research Products (Cambridge, Mass.).

Steroid Compositions

Synthetic analogs of glucocorticoids or preparation of hydrocortisone are available commercially under the names: cortisone acetate, dexamethasone, methylprednisolone acetate, prednisone, hydrocortisone, or prednisolone. Preparations containing these active ingredients are available from various vendors and are commonly administered to cancer patients intravenously or taken orally in tablet form. Triamcinolone acetonide is a derivative of triamcinolone (Muro Pharmaceuticals) approximately eight times more potent than prednisone in animal models of inflammation and is available as an intranasal spray. Loteprednol etabonate is structurally similar to other corticosteroids but the number 20 position ketone group is absent and is used preferentially in occular indications. Medrysone is a synthetic corticosteroid with topical anti-inflammatory and anti-allergic activity. Alclometasone dipropionate, betamethasone, mometasone furoate, halobetasol propionate, fluocinolone acetonide, and flurandrenolide are synthetic corticosteroids (typically fluorinated derivatives) particularly preferred for dermatological applications that can be topically administered. Compositions comprising any of the aforementioned active agents are encompassed by the present invention.

IL-6 Antagonists

As used herein, the term “IL-6 antagonists” refers to a substance which inhibits or neutralizes the angiogenic activity of IL-6. Such antagonists accomplish this effect in a variety of ways. One class of IL-6 antagonists will bind to IL-6 protein with sufficient affinity and specificity to neutralize the angiogenic effect of IL-6. Included in this class of molecules are antibodies and antibody fragments (such as for example, F(ab) or F(ab′)₂ molecules). Another class of IL-6 antagonists are fragments of IL-6 protein, muteins or small organic molecules i.e. peptidomimetics, that will bind to IL-6, thereby inhibiting the angiogenic acitvity of IL-6. The IL-6 antagonist may be of any of these classes as long as it is a substance that inhibits IL-6 angiogenic activity. IL-6 antagonists include IL-6 antibody, IL-6R antibody, an anti-gp130 antibody or antagonist, modified IL-6 such as those disclosed in U.S. Pat. No. 5,723,120, antisense IL-6R and partial peptides of IL-6 or IL-6R.

Anti-IL-6 Antibodies and Agents

Any of the anti-IL-6 antibodies known it the art may be employed in the method of the present invention. Murine monocolonal antibodies to IL-6 are known as in, for example, U.S. Pat. No. 5,618,700 or the antibody known as B-E8 (Diaclone, France) or the antibody referred to as CLB-6/8 capable of inhibiting receptor signaling (Brakenhoff et al, J. Immunol. (1990) (145:561) may be used. To avoid immune response to the antibody which causes adverse effects as well as eliminating the therapeutic action of the antibody, it is desirable to administer a human or close to human antibody scaffold. U.S. Pat. No. 5,856,135 discloses reshaped antibodies to human IL-6 derived from a mouse monoclonal antibody SK2 in which the complementary determining regions (CDR's) from the variable region of the mouse antibody SK2 are transplanted into the variable region of a human antibody and joined to the constant region of a human antibody. A chimerized form of the murine IL-6 monoclonal of the CLB-6/8 murine antibody antibody called cCLB8 was constructed (Centocor, Leiden, The Netherlands) and has been given to multiple myeloma patients (Van Zaanen, et al. 1996 supra). The method of making the resulting antibody from the murine antigen binding domains has been fully described in the applicants' copending application U.S. Ser. No. 10/280,716, hereby incorporated by reference into the present application.

Other process for humanizing of primatizing antibodies raised in non-human species are also suitable for constructing antibodies of the present invention providing the product antibody retains its ability to block IL6 from signaling in the target cell through interaction with its cognate receptor or receptor complex.

Other agents affecting a decrease in IL-6, such as the IL-6 receptor antagonist Sant7 (Tassone et al., Int J Oncol (21) 867-873, 2002) may also be employed.

Anti-Apoptotic Combinations of Steroids and Anti-IL6 Agents

A preferred combination of the present invention uses a standard i.v. or oral steroid preparation such as dexamethosone administered to a patient in combination with a neutralizing anti-IL6 monoclonal antibody.

The neutralizing anti-IL6 monoclonal antibody described herein can be used augment and promote apoptosis in combination with naturally produced corticosteroids or with steroid drug therapy and thereby prevent or impair tumor growth and prevent or inhibit metastases. Additionally, said monoclonal antibody can be used to enhance the anti-inflammatory activity of steroid drugs in diseases amenable to such treatment.

The beneficial effects of the combination of anti-IL-6 monoclonal antibodies with steroids are seen in the tumor response, local control of primary tumor growth and the reduced incidence or rate of metastatic spread. Secondly, the response is more effective than using either of these two agents alone. This combination can be used in a vast array of diseases such as multiple myeloma and edema secondary to primary brain tumors or brain metastasis where effective treatment is yet to be developed. Combining anti-IL-6 and dexamethasone can overcome the resistance to steroid therapy and can also help in reducing the dose of steroid needed to achieve an effect which is essential in minimizing the steroid tapering process; a process necessary to inhibit disease progression and associated symptoms. Finally this combination can decrease resistance to steroids when being used in conjunction with chemotherapy. Further, the combination treatment can have a positive effect on cerebral edema. Currently, steroids are used to treat cerebral edema. Anti-IL-6 therapy could be used to enhance the effect of steroids and decrease side effects observed during steroid tapering.

It is now understood that several signal transduction pathways lead to the stimulus that activates initiation of the apoptotic process. Stimuli that activate these pathways use diverse receptors (JNK, FAS, and the steroid receptors) include ionizing radiation and ceramide in addition to glucocorticoids or analogs (Makin, G. Experts Opin. Ther. Targets 6(1): 73-84, 2002). On the other hand, it has now been demonstrated that the survival signal activated by IL6 includes SHP2 which blocks RAFTK. RAFTK is necessary for the glucocorticoid-induced signal initiating apoptosis (Chauhan, D. et al. J. Biol. Chem. 275(36): 27845-27850, 2000). Thus, the intracellular biochemical basis for at least one mechanism of IL6 antagonism of steroid mediated apoptosis can be understood.

In its broadest sense the invention includes other combinations of agents. For instance, a number of chemotherapy agents are known to induce apoptosis, these include Doxorubicin, arsenic trioxide, retinoids, staurosporin, etoposide, 5-fluorouracil, Paclitaxel, STI571 (Gleevec), Flavoprid, ionizing radiation, Trail, BCL-2 antisense and inhibitors (Makin, Expert Opin Ther Targets (6) 73-84, 2002). Farnesyl transferase inhibitors (Le Gouill et al., Leukemia (16) 1664-7, 2002) may be successfully combined with apoptosis inducing agents, provided that the toxicity profile is acceptable and not additive.

The individual to be treated may be any mammal and is preferably a primate, a companion animal which is a mammal and most preferably a human patient. The amount of monoclonal antibody administered will vary according to the purpose it is being used for and the method of administration.

The anti-IL6 antibodies of the invention of the present invention may be administered by any number of methods that result in an effect in tissue where it is desired to enhance glucocorticoid-induced apoptosis. Further, the anti-IL6 antibodies of the invention may be administered wherever access to body compartments or fluids containing IL6 is achieved. In the case of inflamed, malignant, or otherwise compromised tissues, these methods may include direct application of a formulation containing the antibodies. Such methods include intravenous administration of a liquid composition, transdermal administration of a liquid or solid formulation, oral, topical administration, or interstitial or inter-operative administration. Administration may be affect by the implantation of a device whose primary function may not be as a drug delivery vehicle as, for example, a vascular stent.

Administration may also be oral or by local injection into a tumor or tissue but generally, the monoclonal antibody is administered intravenously. Generally, the dosage range is from about 0.01 mg/kg to about 12.0 mg/kg. This may be as a bolus or as a slow or continuous infusion which may be controlled by a microprocessor controlled and programmabale pump device.

Alternatively, DNA encoding preferably a fragment of said monoclonal antibody may be isolated from hybridoma cells and administered to a mammal. The DNA may be administered in naked form or inserted into a recombinant vector, e.g., vaccinia virus in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Except insofar as any conventional medium is incompatible with the active ingredient and its intended use, its use in any compositions is contemplated.

The formulations may be presented in unit- dose or multi-dose containers, for example, sealed ampules and vials, and may be stored in a freeze-dried (lyophilized) condition requiring only the addition of the sterile liquid carrier, for example, water for injections, immediately prior to use.

Abbreviations

Abs antibodies, polyclonal or monoclonal

aV integrin subunit alpha V

b3 integrin subunit beta 3

bFGF basic fibroblast growth factor

IFN interferon

Ig immunoglobulin

IgG immunoglobulin G

IL interleukin

IL6 interleukin 6

IL-6R interleukin-6 receptor

sIL-6R soluble interleukin-6 receptor

Mab monoclonal antibody

VEGF vascular endothelial growth factor

While having described the invention in general terms, the embodiments of the invention will be further disclosed in the following examples.

### EXAMPLE 1

### Dexamethasone Induced Apoptosis in Multiple Myeloma Cells: Alleviation of IL-6 Mediated Inhibition Using Anti-IL-6 Antibody

Multiple myeloma is a malignant plasma cell disorder that is resistant to conventional therapeutic regimens. IL-6 is known to be a growth and differentiation factor for myeloma cells. Dexamethasone is a glucocorticoid that is part of the standard theraputic regimen for multiple myeloma. Dexamethasone has been reported to induce apoptosis in mutliple myloma cells and cell lines through induction of apoptosis.

Materials and Methods

The cell line RPMI 8226, a human multiple myeloma cell line, was purchased from ATCC (Rockville, Md.). Cells were grown and maintained according to ATCC instructions in complete RPMI medium containing 10% FBS, 1% NEAA, 1% L-glutamine and 1% sodium pyruvate.

Chimeric CLB8 (cCLB8) (Centocor, Malvern, Pa.) was used at three different concentrations in the assay. Another a chimeric human-mouse IgG, c171A, also developed at Centocor was used as a negative control antibody.

Dexamethason-Induced Apoptosis

RPMI 8226 cells (1×10⁶/mL) were incubated for 48 h at 37° C. in a 5% CO2 incubator in RPMI complete medium with or without IL-6 (100 ng/mL), Dexamethasone (1 microM), c171A control antibody (1 microg/mL), or CNTO 328 at three concentrations (1 microg/mL, 100 ng/mL, or 10 ng/mL). After the incubation, cells were harvested and the Tunel assay (Tdt-mediated dUTP-FITC nick end labeling) as disclosed in Gavrieli et al., “Identification of Programmed Cell Death in situ Via Specific Labelling of Nuclear DNA Fragmentation”, J Immunol. Cell Biology 119:493-501, 1992 was used to measure apoptosis with minor modifications. Briefly, after the 48-hour incubation described above, approximately 10⁶ cells were harvested, washed twice, and fixed with 1% paraformaldehyde for 15 minutes. After washing, the cells were permeabilized with 0.1% Triton (Sigma, St. Louis, Mo.) for 5 minutes and washed twice. The labeling reaction was performed in a heating block at 37° C. for 1 hour with 0.3 nM FITC-12-dUTP (Boehringer Mannheim, Indianapolis, Ind.), 2.5 mM CoCI2, 12.5 U Tdt, and 5 microL of 5×Tdt Buffer (Boehringer Mannheim) in a total volume of 50 microL. Cells were analyzed by flow cytometry.

After completing the Tunel assay, cells were washed twice and analyzed on a FACS Calibur flow cytometer (Becton Dickinson Immunocytometry Systems, San Jose, Calif.) equipped with a 15-mW air-cooled 488-nm argon laser. Gating to exclude debris was based upon diminished forward scatter (FSC) and side scatter (SSC). A minimum of 10,000 events was collected per sample and all analyses were performed with CELLQuest software (Becton Dickinson Immunocytometry Systems, San Jose, Calif.).

Results

The results demonstrate that the combination of dexamethasone and cCLB8 is superior to treatment with either agent alone at promoting cellular apoptosis

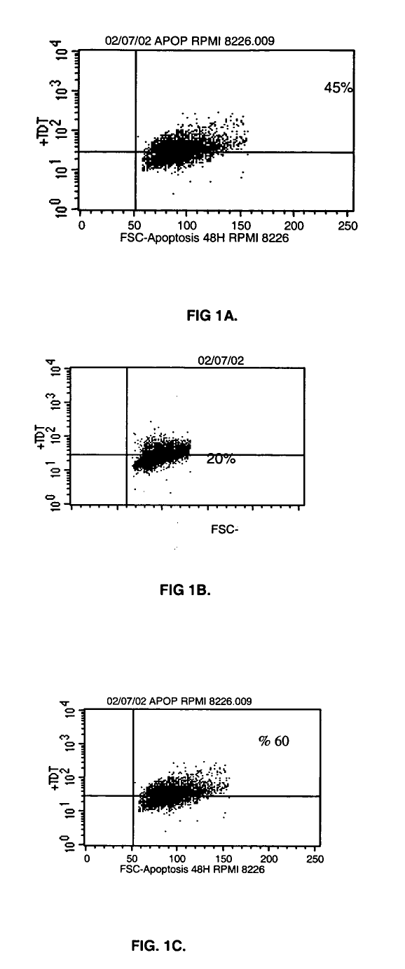

Dexamethasone at 1 microM, induced apoptosis in RPMI 8226 after 48 hrs (FIG. 1A). Dexamethasone induced 45% of cells to undergo apoptosis. At concentrations higher than 100 ng/mL, IL-6 inhibited dexamethasone-induced apoptosis. Dexamethasone in the presence of IL-6 induced only 20% of cells to undergo apoptosis (FIG. 1B). Dexamethasone induced 60% of cells to undergo apoptosis in the presence of both IL-6 and cCLB8 (FIG. 1C).

Table 1 shows the amount of apoptosis exhibited by RPMI 8226 cells subjected to various culture conditions. CCLB8 neutralized the inhibitory effect of IL-6 on dexamethasone-induced apoptosis in a dose dependent manner (P<0.02). The data presented in this table are representative of three experiments and P values were calculated using student T test.

TABLE 1

| | % Apoptosis | |
|---|---|---|
| Treatment | Mean ± SEM | P Value |
| DEX 10-6 | % 46 ± 4 | |
| DEX + IL-6 100 pg | % 27 ± 9 | |
| DEX + IL-6 + CNTO 3281 ug | % 54 ± 2.5 | <0.02 |
| DEX + IL-6 + CNTO 328100 ng | % 45 ± 11 | <0.02 |
| Dex + IL-6 + Control mAb | % 34 ± 9 | <0.04 |

Summary

The experiments described herein demonstrate that effect of IL6 on apoptosis can be reduced by a specific monoclonal antibody that prevents IL6 signaling through a receptor complex which includes gp130. The data demonstrate that IL-6 inhibits dexamethasone-induced apoptosis in multiple myeloma cells. This is the first report to show that the neutralizing effect of cCLB8 on IL-6 inhibition of dexamethasone-induced apoptosis can significantly inhibit tumor cell survival by enhancing glucocorticoid-induced apoptosis and the same levels of apoptosis could not be achieved using either of these agents alone.

What is to be claimed:

1. A method of treating a proliferative disease amenable to treatment by an apoptosis inducing agent in a mammal in need of such treatment which comprises co-administering a steroid in combination with an IL-6 antagonist.

2. The method according to claim 1, in which the IL-6 antagonist is an antibody or a fragment thereof.

3. The method according to claim 2, in which the antibody or fragment binds to IL6.

4. The method according to claim 3, in which the antibody fragment is an Fab, Fab′, or F(ab′)2 fragment or derivative thereof.

5. The method according to claim 3, in which the monoclonal antibody competes with monoclonal antibody cCLB8 for binding to human IL6.

6. The method according to claim 2, in which the monoclonal antibody is administered intravenously

7. The method according to claim 2, in which the monoclonal antibody is administered in the amount of from 0.01 mg/kg to 12.0 mg/kg body weight.

8. The method according to claim 2, in which the monoclonal antibody is administered in a bolus dose followed by an infusion of said antibody.

9. The method according to claim 1, in which the mammal is a human patient.

10. The method according to claim 1 in which the steroid is selected from the group consisting of cortisone acetate, dexamethasone, methylprednisolone acetate, hydrocortisone, prednisone, or prednisolone.

11. The method according to claim 1, in which the proliferative disease is cancer.

12. The method of claim 11, wherein the disease is a disease selected from the group consisting of cancer metastasis, multiple myeloma, seborrheic dermatitis, acne and arthritis.

13. A method for inhibiting tumor growth in a mammal in need thereof comprising administering to the mammal in conjunction with a corticosteroid therapeutic, a monoclonal antibody or fragment thereof which prevents IL6 activation of signaling through membrane bound receptors in an amount effective to inhibit the growth of said tumor.

14. A method for preventing metastases in a mammal comprising administering to the mammal in conjunction with a corticosteroid therapeutic, a monoclonal antibody or fragment thereof which prevents IL6 activation of signaling through membrane bound receptors in an amount effective to prevent metastases in said mammal.

15. A method for treating cerebral edema in a mammal comprising administering to the mammal a corticosteroid in combination with an IL-6 antagonist in an amount effective to treat cerebral edema in said mammal.

16. A method of any of claims 1, 12, 13, 14 or 15 wherein the antibody is cCLB8 or a fragment thereof.
